SEQUENCE LISTING (2 sequences)

SEQ ID NO: 1 - DNA, 19 bp, Artificial Sequence
  SEQ ID NO 1 Cobitolimod
  misc_feature (1)..(4): phosphorothioate linkage between bases 1-2, 2-3 and 3-4
  misc_feature (16)..(19): phosphorothioate linkage between bases 16-17, 17-18 and 18-19
ggaacagttcgtccatggc

SEQ ID NO: 2 - DNA, 19 bp, Artificial Sequence
  SEQ ID NO 2 synthesized oligonucleotide
ggaacagttcgtccatggc

(19) United States
(12) Patent Application Publication
(10) Pub. No.: US 2022/0218736 A1
(43) Pub. Date: Jul. 14, 2022

(54) NEW THERAPY

(71) Applicant: INDEX PHARMACEUTICALS AB, Solna (SE)

(72) Inventors: Arezou Zargari, Solna (SE); Pernilla Sandwall, Solna (SE); Charlotte Admyre, Solna (SE); Thomas Knittel, Solna (SE); Peter Zerhouni, Solna (SE)

(21) Appl. No.: 17/290,582

(22) PCT Filed: Nov. 14, 2019
(86) PCT No.: PCT/EP2019/081377

§ 371 Date: Apr. 30, 2021

(30) Foreign Application Priority Data
Nov. 14, 2018 (GB) 1818579.3

(51) Int. Cl. A61K 31/7115; A61P 1/00; C12N 15/113

(52) U.S. Cl. CPC A61K 31/7115; C12N 15/113; A61P 1/00

(57) ABSTRACT

The present invention provides an oligonucleotide for use in preventing the recurrence of active ulcerative colitis in a patient, wherein the oligonucleotide comprises the sequence 5′-GGAACAGTTCGTCCATGGC-3′ (SEQ ID NO:2).

### RELATED APPLICATIONS

The present application is a U.S. national phase application under 35 U.S.C. § 371 of

International Application No. PCT/EP2019/081377, filed on Nov. 14, 2019 and published as WO 2020/099585 A1 on May 22, 2020, which claims priority to GB Application No. 1818579.3, filed on Nov. 14, 2018. The content of each of these related applications is incorporated herein by reference in its entirety.

### REFERENCE TO SEQUENCE LISTING

The present application includes a Sequence Listing in electronic format. The Sequence Listing is provided as a file entitled Sequence_Listing_63CZ-312122-US, created Sep. 28, 2021, which is 1 kilobyte in size. The information in the electronic format of the Sequence Listing is incorporated herein by reference in its entirety.

### FIELD OF THE INVENTION

The present invention relates to new therapies for preventing the recurrence of active ulcerative colitis (UC), involving the administration of an oligonucleotide, especially cobitolimod.

### BACKGROUND OF THE INVENTION

Ulcerative colitis (UC) is a disease characterized by chronic inflammation of the rectal and colonic mucosa, affecting the innermost lining in the first stage. The disease is recurrent, with both active and inactive stages that differ in pathology, symptoms and treatment. The underlying cause of ulcerative colitis is not understood, nor is it known what triggers the disease to recur between its inactive and active forms (Irvine, E. J. (2008) Inflamm Bowel Dis 14(4): 554-565). The highest annual incidence of UC as reported in 2012 was 24.3 per 100,000 person-years in Europe, 6.3 per 100,000 person- years in Asia and the Middle East, and 19.2 per 100,000 person-years in North America, with a prevalence of 505 cases per 100,000 persons in Europe and 249 cases per 100,000 persons in North America (Molodecky N A, Soon I S, Rabi D M, et al. Gastroenterology. 2012:142(1):46- 54).

Symptoms of active ulcerative colitis include progressive loose stools with blood and increased frequency of bowel movements. Active mucosal inflammation is diagnosed by endoscopy. The stools contain pus, mucous and blood and are often associated with abdominal cramping with urgency to evacuate (tenesmi). Diarrhoea may have an insidious onset or, more rarely, start quite suddenly. In severe cases the symptoms may include fever and general malaise. In severe stages, deep inflammation of the bowel wall may develop with abdominal tenderness, tachycardia, fever and risk of bowel perforation. Furthermore, patients with ulcerative colitis may suffer extra intestinal manifestations such as arthralgia and arthritis, erythema nodosum, pyoderma gangrenosum and inflammation in the eyes. In the case of remission or inactive ulcerative colitis, patients are usually free of bowel symptoms.

The extent of inflamed and damaged mucosa differs among patients with ulcerative colitis. Ulcerative colitis that affects only the rectum is termed ulcerative proctitis. The condition is referred to as distal colitis when inflammatory changes are present in the left side of the colon up to the splenic flexure. In extensive ulcerative colitis the transverse colon is also affected, and pancolitis designates a disease involving the entire colon.

Active mucosal inflammation is diagnosed by endoscopy and is characterized by a loss of vascular patterning, oedema, petechia, spontaneous bleeding and fibrinous exudates. The endoscopic picture is that of continuous inflammation, starting in the rectum and extending proximally to a variable extent into the colon. Biopsies obtained at endoscopy and subjected to histological examination help to diagnose the condition. Infectious causes, including clostridium difficile, campylobacter, Salmonella and Shigella, may mimic ulcerative colitis and can be excluded by stool cultures.

The medical management of ulcerative colitis is divided into treatment of active disease and maintenance of remission.

Whilst treatment of active phases of the disease is important, the prevention or prophylaxis of relapse into or recurrence of further active phases is crucial for long-term management of the disorder. Maintenance treatment is the use of a drug over a prolonged period of time. Such maintenance treatment can be used, for example, to reduce the frequency of active phases i.e. to keep patients in an inactive stage of the disease.

Examples of preventing active ulcerative colitis from reoccurring include maintaining a low disease activity index, a normal body weight or a low histopathological score (for example below 3) and avoiding a relapse into an active ulcerative colitis state by maintaining a remission state.

A number of treatments are available for inducing remission of active ulcerative colitis. Treatment of patients with active ulcerative colitis aims to reduce inflammation and promote colon healing and mucosal recovery. In milder cases the disease may be controlled with conventional drugs including aminosalicylates, such as sulfasalazine and 5-aminosalicylic acid (5-ASA) (Sutherland, L., F. Martin, S. Greer, M. Robinson, N. Greenberger, F. Saibil, T. Martin, J. Span, E. Prokipchuk and L. Borgn (1987) Gastroenterology 92: 1894-1898), and glucocorticosteroids (GCS) (Domenech, E., M. Manosa and E. Cabre (2014). Dig Dis 32(4): 320-327). Distal disease, limited to the rectum and the recto-sigmoid area, is usually brought into remission using rectal formulations. When the extent is more proximal and/or the symptoms more severe, oral GCS is also needed.

For patients who become refractory or intolerant to GCS, or unwilling to use GCS, and suffer from severe or moderately severe attacks of ulcerative colitis, immunomodulatory agents such as cyclosporine, 6-mercaptopurine [6-MP], azathioprine [AZA], methotrexate and tacrolimus are typically used. These immunomodulatory agents may also be used with patients who become refractory or intolerant to aminosalicylates, or unwilling to use aminosalicylates, and suffer from severe or moderately severe attacks of ulcerative colitis.

Further treatment options for ulcerative colitis include biologic agents (Fausel, R. and A. Afzali (2015) Ther Clin Risk Manag 11: 63-73). Three TNF-a inhibitors currently approved for the treatment of moderate to severe ulcerative colitis are infliximab, adalimumab, and golimumab. All three carry potential risks associated with their use, and should be avoided in certain patients, e.g. those with uncontrolled infections, advanced heart failure, neurologic conditions and in patients with a history of malignancy, due to a potential risk of accelerating the growth of a tumour. Other potential adverse effects of TNF-α inhibitor therapy include neutropenia, hepatotoxicity, serum sickness, leukocytoclastic vasculitis, rash including psoriasiform rash, induction of autoimmunity, and injection or infusion site reactions, including anaphylaxis, convulsions, and hypotension. Further treatment options for ulcerative colitis include anti-integrin antibodies, such as vedolizumab, and JAK inhibitors, such as tofacitinib.

It is unpredictable whether a therapeutic agent that is known to be effective in inducing remission of active ulcerative colitis will also be beneficial or suitable for maintaining remission.

Indeed, few of the agents recommended for treatment of active episodes of ulcerative colitis are suitable for long-term maintenance therapy. Thus, GCS are generally not recommended for maintenance therapy since there are significant side effects associated with their long-term use, and the possibility of developing steroid dependent disease.

Maintenance treatment with systemic GCS has been explicitly advised against in the literature (Prantera, C. and S. Marconi (2013) Therap Adv Gastroenterol 6(2): 137-156). Patients with moderate to severe symptoms may derive some benefits from immunosuppressant agents (e.g. cyclosporine, 6-mercaptopurine [6-MP], azathioprine [AZA], methotrexate [MTX] and tacrolimus). However, the use of these agents is limited as induction treatments due to a slow onset of action (3 to 6 months) and as maintenance therapies due to adverse events (AEs), including bone marrow suppression, infections, hepatotoxicity, pancreatitis, and malignancies. (Kombluth A, Sachar D B. Am J Gastroenterol., 2010; 105(3):501-23; quiz 524 and Beaugerie L, Brousse N, Bouvier A M, et al. Lancet. 2009; 374(9701):1617-25). As to biologic agents, a large proportion of patients (>50%) have been found to lose response to biologic TNF-α inhibitor agents over time (Fausel, R. and A. Afzali (2015) Ther Clin Risk Manag 11: 63-73). Combined with their serious side effects, this means such biologic agents do not therefore represent an ideal long-term maintenance solution.

Notwithstanding the limitations discussed above, active agents that are currently used as maintenance therapies for ulcerative colitis include aminosalicylates, 6-MP, AZA, TNF-α inhibitors [such as infliximab, adalimumab and golimumab], vedolizumab and tofacitinib.

In the absence of appropriate long-term maintenance therapy, relapses of patients in remission become inevitable in the majority of cases. Ulcerative colitis may become chronically active, and essentially refractory to available treatments. The only remaining course of action in such cases is colectomy. A total colectomy is a potentially curative option in severe ulcerative colitis, but is a life-changing operation that entails risks as complications, such as pouch failure, pelvic sepsis, infertility in women, and nocturnal faecal soiling, may follow. These complications may ultimately lead to mortality in around 0.5% of cases (Ferrante M, et al, Inflamm Bowel Dis 2008; 14:20-8). It is therefore desirable to find a long-term maintenance therapy in order to prevent relapsing of ulcerative colitis.

Cobitolimod (Kappaproct/DIMS0150) is a modified single strand DNA-based synthetic oligodeoxyribonucleotide of 19 bases in length having the sequence 5′-G*G*A*ACAGTTCGTCCAT*G*G*C-3′ (SEQ ID NO:1), wherein the CG dinucleotide is unmethylated, and wherein an asterisk (*) indicates a phosphorothioate linkage in the sequence, that functions as an immunomodulatory agent by targeting the Toll-like receptor 9 (TLR9) present in immune cells. These immune cells (i.e., B-cells and plasmacytoid dendritic cell (pDCs) reside in high abundance on mucosal surfaces, such as colonic and nasal mucosa. The immune system is the key mediator of the changes involved in ulcerative colitis. The mucosa of the colon and rectum of patients with ulcerative colitis contains active immune cells, which results in damage to the tissue. Cobitolimod may be topically administered in the region of inflammation, which places the drug in close contact with a high number of intended target cells, ensuring that the drug will reach an area rich in TLR9 expressing cells. The activation of these cells by cobitolimod induces various cytokines, such as type I interferons and interleukin 10 (IL-10) which are believed to be important factors for the clinical effect of cobitolimod.

A range of non-clinical safety studies have been conducted with cobitolimod, as well as four clinical trials to assess cobitolimod's ability to induce remission in active ulcerative colitis patients. Overall, data on cobitolimod support a positive benefit-risk assessment for patients with ulcerative colitis. Cobitolimod is safe and well tolerated and has been shown to be effective at clinical response and inducing remission in patients with active ulcerative colitis, as well as in symptomatic and endoscopic remission in patients with severe active treatment refractory ulcerative colitis or moderate to severe active treatment refractory ulcerative colitis.

Given the paucity of safe and efficacious maintenance therapies for preventing recurrence of active ulcerative colitis, and the problems associated with known therapies, there exists a need for the development of new maintenance therapies with better efficacy, safety and tolerability profiles.

### SUMMARY OF THE INVENTION

It has now surprisingly been found that administration of cobitolimod can be used to prevent recurrence of or relapse into active ulcerative colitis and the debilitating symptoms associated therewith without the side effects of known ulcerative colitis therapies.

The present invention therefore provides an oligonucleotide for use in preventing the recurrence of active ulcerative colitis in a patient, wherein the oligonucleotide comprises the sequence 5′-GGAACAGTTCGTCCATGGC-3′ (SEQ ID NO:2).

The present invention also provides a pharmaceutical composition comprising an oligonucleotide as defined herein, together with one or more pharmaceutically acceptable carriers, for use in preventing the recurrence of active ulcerative colitis as defined herein in a patient as defined herein.

The present invention also provides a method of preventing the recurrence of active ulcerative colitis as defined herein, in a patient as defined herein, comprising administering to said patient an oligonucleotide as defined herein or a composition as defined herein.

The present invention also provides use of an oligonucleotide as defined herein, for the manufacture of a medicament for preventing the recurrence of active ulcerative colitis in a patient as herein defined.

In preferred embodiments, the oligonucleotide has the sequence 5′- G*G*A*ACAGTTCGTCCAT*G*G*C-3′ (SEQ ID NO:1), wherein the CG dinucleotide is unmethylated. Thus, in preferred embodiments, the oligonucleotide is cobitolimod. Phosphorothioate linkages are indicated by asterisks (*) in the sequence.

### BRIEF DESCRIPTION OF THE FIGURES

FIG. 1 shows the percentage of patients in sustained symptomatic remission at week 4 following dosing with 30 mg cobitolimod at weeks 0 and 4, and how that percentage changes for that group of patients at weeks 8 and 12.

FIG. 2 shows the treatment course of patient p12 in example 2, showing the colitis activity index [CAI] against the time in months. Numbers on the line indicate a particular CAI score at a certain timepoint. A CAI score with a value above the upper dashed line shows that the patient has a severe flare in the disease and below the lower dashed line that the patient is in clinical remission. The line without numbers shows the course of the steroid treatment with the amount of Decortin [mg] was given per day. A grey arrow indicates a dosing visit (26 Mar. 2010, 28 Apr. 2010, 26 May 2010, 25 Aug. 2010, 22 Sep. 2010, 19 Oct. 2010), a black arrow a visit where a CAI score could be established (1 Apr. 2010, 5 May 2010, 19 May 2010, 23 Jun. 2010, 12 Jul. 2010, 1 Sep. 2010, 25 Oct. 2010, 24 Nov. 2010, 9 Mar. 2011) and the grey arrow on the right hand side shows the status on 24 Mar. 2011 when the treating physician last contacted all the patients to follow-up on the treatments.

FIG. 3 is a photo of representative colons from each group of animals in example 3.

FIG. 4 shows the colon length (in cm) for each treatment group in example 3 and is expressed as Mean±SD for each group. Significance [*: p<0.05, ***: p<0.005, and ****: p≤0.001] was determined by One-Way ANOVA with Tukey's multiple comparisons test.

FIG. 5 shows the colon weight/length ratio for each treatment group in example 3 and is expressed as Mean±SD for each group. Significance [*: p<0.05, ***: p<0.005, and ****: p≤0.001] was determined by One-Way ANOVA with Tukey's multiple comparisons test.

### DETAILED DESCRIPTION OF THE INVENTION

All patents, patent applications, and publications cited herein are hereby incorporated by reference in their entirety.

As used herein, the term “patient” typically refers to a human patient. Patients may, however, be other vertebrate animals, such as mammals. The terms “subject” and “patient” are used interchangeably herein.

As used herein, the words “treatment” and “treating” are to be understood as embracing treatment and/or amelioration and/or prevention of or reduction in aggravation/worsening of symptoms of a disease or condition as well as treatment of the cause of the disease or condition, and may include reversing, reducing, or arresting the symptoms, clinical signs, and underlying pathology of a condition in a manner to improve or stabilise a subject's condition.

Reference to “prevention” and “preventing” (recurrence of) a disease or condition embraces prophylaxis and/or inhibition of the disease or condition. The term “preventing” is art-recognized, and when used in relation to a condition, such as ulcerative colitis or its associated symptoms, is well understood in the art, and includes administration of a drug, e.g. an oligonucleotide, and/or composition which reduces the frequency of, or delays the onset of, symptoms of a medical condition in a subject relative to a subject which does not receive the drug or composition.

The disease ulcerative colitis is well known to one skilled in the art. One skilled in the art will be well acquainted with the concepts of the active and remission phases of the disease. Ulcerative colitis manifests as active phases of the disease characterised by typical symptoms and, typically following treatment of the active phase of the disease, a remission phase essentially free of symptoms. Typical symptoms indicative of the active phase of ulcerative colitis include blood in stool and increased stool frequency. The active phase of ulcerative colitis can be diagnosed using a variety of different scoring systems, as discussed below, including the Mayo and Modified Mayo scoring systems.

In the context of the present invention, reference to preventing the recurrence of active ulcerative colitis is intended to refer to preventing recurrence of the active phase of the disease, i.e. maintaining a patient in remission and providing a maintenance therapy.

Thus, the present invention relates to the use of an oligonucleotide as defined herein to prevent the recurrence of or relapse into the active phase of ulcerative colitis (i.e. providing a maintenance therapy).

Thus, prevention of the recurrence of or relapse into active ulcerative colitis includes, for example, delaying the onset (prolonging the time between episodes) or reducing the number (incidence), frequency, severity or duration of one or more of the active phases of ulcerative colitis in a treated population versus a control population untreated with the oligonucleotide, e.g., by a statistically and/or clinically significant amount. The patient thus typically remains in clinical remission. Mucosal healing (as generally assessed by endoscopic appearance) in the patient is also typically maintained. Clinical remission and mucosal healing are thus preferably maintained/sustained in patients.

It is of clinical benefit to prevent the recurrence of or relapse into active ulcerative colitis for as long a period of time as possible to ensure the best quality of life for the patient. Remission may be defined as absence or minimal symptoms as discussed herein for at least one month. Given that ulcerative colitis is a chronic condition that cannot be cured, typically prevention of recurrence of or relapse into refers to prevention of recurrence/relapse for extended periods of time, typically for at least 6 months, preferably for at least 1 year or 2 years, for example for at least 5 years or 10 years. Ideally, recurrence/relapse is prevented indefinitely.

Remission may be defined as clinical remission, symptomatic remission, endoscopic remission and/or histopathological remission. Remission may also be patient-reported remission, which generally corresponds to symptomatic remission.

Clinical remission may be as defined herein with reference to the Clinical Activity (Rachmilewitz) Index (CAI), the Mayo Score or the Modified Mayo Score. Symptomatic remission (or patient-reported remission) may be defined as no blood in stool, with a weekly stool frequency of less than 35. Endoscopic remission arises where mucosal healing is observed during endoscopy (e.g. a Mayo sub-score of less than 2). Histopathological remission may be defined as a Geboes score of less than 2 or a Nancy Index of grade 0 or 1. Deep remission is where both endoscopic remission and histopathological remission are observed.

In one aspect, the oligonucleotide for use according to the present invention is used to maintain a patient in clinical remission. In another aspect, the oligonucleotide for use according to the present invention is used to maintain a patient in symptomatic remission (or patient-reported remission). In another aspect, the oligonucleotide for use according to the present invention is used to maintain a patient in endoscopic remission (i.e. with mucosal healing maintained). In another aspect, the oligonucleotide for use according to the present invention is used to maintain a patient in histopathological remission.

Preferably, the oligonucleotide for use according to the present invention is used to maintain a patient in (a) clinical remission and symptomatic remission, or (b) clinical remission and endoscopic remission, or (c) clinical remission and histopathological remission, or (d) symptomatic remission and endoscopic remission, or (e) in symptomatic remission and histopathological remission, or (f) in endoscopic remission and histopathological remission. More preferably, the oligonucleotide for use according to the present invention is used to maintain a patient in (a) clinical remission, symptomatic remission and endoscopic remission, or (b) clinical remission, symptomatic remission and histopathological remission, or (c) clinical remission, endoscopic remission, and histopathological remission, or (d) symptomatic remission, endoscopic remission and histopathological remission. Most preferably the oligonucleotide for use according to the present invention is used to maintain a patient in clinical remission, symptomatic remission, endoscopic remission and histopathological remission.

The oligonucleotide for use according to the present invention may be used to maintain a patient in steroid-free remission. In this case, the patient is kept in remission without the need for concomitant administration of steroids.

Reference to “recurrence” or “relapse” indicates that a patient has previously suffered from active ulcerative colitis. A patient “recurs” or “relapses” when, having experienced a period of remission or recovery, the patient experiences one or more symptoms indicative of active ulcerative colitis. Typically, a patient has experienced one or more symptoms associated with active ulcerative colitis, as defined herein, and essentially symptom free periods (“remission” or “recovery”) between active phases, usually following treatment. The administration of the oligonucleotide typically extends those symptom free periods for as long as possible, for example for a particular period of time, as defined above.

Thus, the oligonucleotide as defined herein is for use in a patient that has previously experienced active ulcerative colitis. The oligonucleotide as defined herein is for use in preventing the recurrence of active ulcerative colitis in the patient. Typically, the patient has been diagnosed as suffering from ulcerative colitis. Typically, the patient has previously experienced one or more active phases of ulcerative colitis as defined herein and the oligonucleotide for use in the present invention acts to prevent further such active phases of any duration.

Typically, a patient has experienced one or more symptoms associated with mild, moderate or severe active ulcerative colitis. The oligonucleotide may be for use in a patient that has previously experienced mild, moderate or severe active ulcerative colitis. The oligonucleotide may be for use in preventing the recurrence of mild, moderate or severe active ulcerative colitis in a patient. For instance, the oligonucleotide may be for use in a patient that has previously experienced mild active ulcerative colitis. For instance, the oligonucleotide may be for use in a patient that has previously experienced moderate active ulcerative colitis. For instance, the oligonucleotide may be for use in a patient that has previously experienced severe active ulcerative colitis. The phrases mild, moderate and severe may be as described herein with reference to any of the disease indices mentioned.

Patients with ulcerative colitis typically present with a spectrum of disease severity ranging from remission to severely active. Clinical assessment can be used to classify ulcerative colitis patients into 4 disease activity subgroups as defined in D′Haens, Gastroenterology 2007; 132: 763-786, the entirety of which is incorporated herein by reference: (1) remission (≤2 or 3 stools/ day, without the presence of blood and/or pus in the stools, with no systemic symptoms); (2) mildly active disease (3 or 4 stools/day and/or presence of blood and/or pus in the stools less than daily, with no systemic symptoms of fever or weight loss); (3) moderately active disease (>4 stools/day and/or daily presence of blood and/or pus) with minimal systemic symptoms; and (4) severely active disease (>6 bloody stools/day, and evidence of toxicity, as demonstrated by fever, tachycardia, anemia, or an erythrocyte sedimentation rate ESR). Other scoring systems as described herein can be used to categorise disease severity in patients as well.

Typically, the patient treated in accordance with the present invention is not suffering from active ulcerative colitis. Typically, the patient treated in accordance with the present invention is not suffering from active ulcerative colitis as determined by the Mayo Score or Modified Mayo Score, preferably the Modified Mayo Score. The EMA indicated in its “Guidelines on the development of new medicinal products for the treatment of Ulcerative Colitis” dated 28 Jun. 2018 that the Modified Mayo Score is recommended. Similarly, the FDA indicated in “Ulcerative Colitis: Clinical Trial Endpoints—Guidance for Industry” issued in draft in August 2016 that the Modified Mayo Score is recommended.

Thus, the oligonucleotide of the invention is for administration to a patient who is in remission as described herein, i.e. a patient in which the ulcerative colitis is in an inactive state.

Patients who are in a remission phase of ulcerative colitis typically have a Modified Mayo Score ≤2, with a rectal bleeding sub-score of 0, a stool frequency sub-score of 0 or 1, and an endoscopy sub-score of 0 or 1.

Induction of response or remission in ulcerative colitis patients may be determined in accordance with one or more standard disease indices. Typical disease indices include but not limited to the ones mentioned below; (i) disease activity determined by clinical and biochemical disease activity, (ii) disease activity determined by endoscopic disease activity, (iii) disease activity determined by composite clinical and endoscopic disease activity indices, (iv) quality of life, (v) histologic disease activity. These indices are discussed in D'Haens, Gastroenterology 2007; 132: 763-786, the entirety of which is incorporated herein by reference.

Indices based on disease activity determined by clinical and biochemical disease activity include the Truelove and Witts Severity Index; Powell-Tuck (St. Mark's) Index; Clinical Activity (Rachmilewitz) Index (CAI); Activity (Seo) Index; Physician Global Assessment; Lichtiger (Modified Truelove and Witts Severity) Index; Investigators Global Evaluation; Simple Clinical Colitis Activity Index; Improvement Based on Individual Symptom Scores; Ulcerative Colitis Clinical Score; and Patient-defined remission. These indices are discussed in D'Haens (ibid).

Indices based on disease activity determined by endoscopic disease activity include the

Truelove and Witts Sigmoidoscopic Assessment; Baron score; Powell-Tuck Sigmoidoscopic Assessment; Endoscopic (Rachmilewitz Endoscopic) Index; Sigmoidoscopic Index; Sigmoidoscopic Inflammation Grade Score; Mayo Score Flexible Proctosigmoidoscopy Assessment; Sutherland Mucosal Appearance Assessment; and Modified Baron Score. These indices are discussed in D'Haens (ibid).

Indices based on disease activity determined by composite clinical and endoscopic disease activity indices include the Mayo Score (Mayo Clinic Score/Disease Activity Index); Modified Mayo Score and Sutherland Index (Disease Activity Index/UC Disease Activity Index). Mayo Score and Sutherland Index are discussed in D'Haens (ibid).

Indices based on quality of life include the Rating Form of IBD Patient Concerns; and the Inflammatory Bowel Disease Questionnaire (IBDQ). These indices are discussed in D'Haens (ibid). Indices based on histologic disease activity include those discussed in D'Haens (ibid) such as Geboes Index and Riley Index and further indices such as Nancy Index and Robarts Index.

Preferred indices for assessing ulcerative colitis patients include the Clinical Activity (Rachmilewitz) Index, Mayo Score and Modified Mayo Score.

The Clinical Activity (Rachmilewitz) Index is an index taking into account 7 variables: number of stools, blood in stools, investigator's global assessment of symptomatic state, abdominal pain or cramps, temperature due to colitis, extraintestinal manifestations, and laboratory findings. This is discussed further in D'Haens (ibid) and Rachmilewitz D., BMJ 1989; 298: 82-86, the entirety of which is incorporated herein by reference. Determination of the Clinical Activity (Rachmilewitz) Index produces a score for a patient ranging from 0 to 29 points (higher scores meaning more severe disease).

Clinical remission may be considered as a Clinical Activity (Rachmilewitz) Index score ≤4 points. Response as determined by the Clinical Activity (Rachmilewitz) Index means the patient has a lower score after treatment than before treatment.

The Mayo Score is an index taking into account 4 items: stool frequency, rectal bleeding, findings of flexible proctosigmoidoscopy , and Physician's Global Assessment (PGA). This is discussed further in D'Haens (ibid) and Schroeder K W et al, N Engl J Med 1987; 317: 1625-1629, the entirety of which is incorporated herein by reference. Determination of the Mayo Score produces a score ranging from 0 to 12 points (higher scores meaning more severe disease). In addition to the four specific items, a patient's functional assessment is also measured that is not meant to be included in the 12-point index calculation but should be used as a measure of general well-being when determining the PGA sub-score.

Mayo scoring for each of the 4 items is determined as set out in the Table below.

| | | | Physician's | |
|---|---|---|---|---|
| | Stool | Rectal | global | Colonoscopy/ |
| Score | frequency^{b} | Bleeding^{c} | assessment^{d} | sigmoidoscopy finding |
| 0 | Normal | No blood | Normal or | Normal or inactive |
| | number of | seen | no disease | disease |
| | stools for | | | |
| | this patient | | | |
| 1 | 1 to 2 stools | Streaks of | Mild | Mild disease |
| | more than | blood with | disease | (erythema, decreased |
| | normal | stool less | | vascular pattern, |
| | | than half | | mild friability) |
| | | the time | | |
| 2 | 3 to 4 stools | Obvious | Moderate | Moderate disease |
| | more than | blood with | disease | (marked erythema, |
| | normal | stool most | | lack of vascular |
| | | of the time | | pattern, friability, |
| | | | | erosions) |
| 3 | 5 or more | Blood | Severe | Severe disease |
| | stools more | alone | disease | (spontaneous bleeding, |
| | than normal | passed | | ulceration) |

^{b}Each patient serves as his or her own control to establish the degree of abnormality of the stool frequency.
^{c}The daily bleeding score represents the most severe bleeding of the day.
^{d}The physician's global assessment acknowledges the 3 other criteria, the patient's daily record of abdominal discomfort and general sense of well-being, and other observations, such as physical findings and the patient's performance status.

Remission (complete response) according to the Mayo Score may be defined as complete resolution of (1) stool frequency (normal stool frequency), (2) rectal bleeding (no rectal bleeding), (3) patient's functional assessment score (generally well), (4) endoscopy findings (normal), and a PGA score of 0. Partial response as determined by Mayo Score typically requires improvement (a minimum 1-point decrease from baseline) in the PGA score and improvement in at least one other clinical assessment (stool frequency, rectal bleeding, patient's functional assessment, endoscopy findings) and no worsening in any other clinical assessment.

Alternatively, clinical remission may be defined as a Mayo Score of 0 and clinical improvement (response) as a decrease from baseline in the Mayo Score ≥3 points.

Alternatively, clinical remission may be defined as a Mayo Score of 0 and clinical improvement (response) as a decrease from baseline in the Mayo Score ≥3 points (or a decrease of ≥2 points if the baseline Mayo Score was <3 points).

Alternatively, remission as determined by Mayo Score may be defined as requiring sub-scores of 0 for both endoscopy and rectal bleeding and a score of 0 or 1 for stool frequency and PGA sub-scores. Response may be defined as a decrease from baseline in the Mayo Score ≥3 points; clinical response may be defined as a decrease from baseline in the Mayo Score (without the endoscopy sub-score, also known as a Partial Mayo Score) ≥2 points, and endoscopic response may be defined as a decrease from baseline in the endoscopic sub-score ≥1 point.

Alternatively, clinical remission may be defined as a total Mayo score of ≤2 points with no individual sub-score >1 point, clinical response may be defined as a decrease from baseline in the total Mayo score ≥3 points and ≥30% and a decrease in the rectal bleeding sub-score ≥1 point or an absolute rectal bleeding sub-score of 0 or 1, and mucosal healing may be defined as an absolute endoscopy sub-score of 0 or 1.

Typically, patients having active ulcerative colitis have a Mayo Score >2. Patients who are in a remission phase of ulcerative colitis typically have a Mayo Score ≤2.

Modified Mayo Score is related to the Mayo Score, which is defined above. Modified Mayo Score differs from Mayo Score, for example, in that the endoscopy scoring takes less account of friability. The scoring table for this Modified Mayo Score is as set out below. An alternative Modified Mayo Score may also be a Mayo Score without the PGA sub-score.

Modified Mayo score is typically assessed in accordance with known methods (Schroder K W, Tremaine W J, Ilstrup D M. (1987) N Engl J Med 317(26):1625-9).

The Modified Mayo without friability in grade 1 of the rectal bleeding sub-score ranges from 0-12, with higher scores indicating more severe disease.

Modified Mayo score without friability in grade 1 of the rectal bleeding sub-score can usually be assessed using the following scoring system. Use of such a system to assess a patient would be routine for one of skill in the art.

| | | | Physician's | |
|---|---|---|---|---|
| | Stool | Rectal | global | Colonoscopy/ |
| Grade | frequency^{b} | Bleeding^{c} | assessment^{d} | sigmoidoscopy finding |
| 0 | Normal | No blood | Normal or | Normal or inactive |
| | number of | seen | no disease | disease |
| | stools for | | | |
| | this patient | | | |
| 1 | 1 to 2 stools | Streaks of | Mild | Mild disease |
| | more than | blood with | disease | (erythema, decreased |
| | normal | stool less | | vascular pattern) |
| | | than half | | |
| | | the time | | |
| 2 | 3 to 4 stools | Obvious | Moderate | Moderate disease |
| | more than | blood with | disease | (marked erythema, |
| | normal | stool most | | lack of vascular |
| | | of the time | | pattern, friability, |
| | | | | erosions) |
| 3 | 5 or more | Blood | Severe | Severe disease |
| | stools more | alone | disease | (spontaneous bleeding, |
| | than normal | passed | | ulceration) |

^{b}Each patient serves as his or her own control to establish the degree of abnormality of the stool frequency.
^{c}The daily bleeding score represents the most severe bleeding of the day.
^{d}The physician's global assessment acknowledges the 3 other criteria, the patient's daily recollection of abdominal discomfort and general sense of well-being, and other observations, such as physical findings and the patient's performance status. The physician's global assessment may be omitted from the overall score.

Remission and response values for the Modified Mayo Score are as set out above for the Mayo Score. Modified Mayo Score is typically assessed in accordance with the FDA's draft guidance document “Ulcerative Colitis: Clinical Trial Endpoints Guidance for Industry” found at http://www.fda.gov/downloads/Drugs/GuidanceComplianceRegulatoryInformation/Guidances/UCM515143.pdf

Alternatively, Modified Mayo Score may differ from Mayo Score in that the Colonoscopy/sigmoidoscopy scoring takes less account of friability and also in that Physician's Global Assessment is not determinative. Thus, the scoring table for the Modified Mayo Score may also be as follows.

| | Stool | Rectal | Colonoscopy/sigmoidoscopy |
|---|---|---|---|
| Score | frequency^{b} | Bleeding^{c} | finding |
| 0 | Normal | No blood | Normal or inactive disease |
| | number of | seen | |
| | stools for this | | |
| | patient | | |
| 1 | 1 to 2 stools | Streaks of | Mild disease (erythema, |
| | more than | blood with | decreased vascular pattern) |
| | normal | stool less | |
| | | than half the | |
| | | time | |
| 2 | 3 to 4 stools | Obvious | Moderate disease (marked |
| | more than | blood with | erythema, lack of vascular |
| | normal | stool most of | pattern, friability, erosions) |
| | | the time | |
| 3 | 5 or more | Blood alone | Severe disease (spontaneous |
| | stools more | passed | bleeding, ulceration) |
| | than normal | | |

^{b}Each patient serves as his or her own control to establish the degree of abnormality of the stool frequency.
^{c}The daily bleeding score represents the most severe bleeding of the day.

Remission and response values for this alternative Modified Mayo Score are typically as set out above for the Mayo Score. Alternatively, remission may be defined in accordance with this alternative Modified Mayo Score by sub-scores of i) rectal bleeding of 0, ii) stool frequency of 0 or 1 (with at least one point decrease from Baseline, Week 0), and iii) endoscopy score of 0 or 1 (excluding friability). Clinical response may be defined as clinical remission or a decrease from baseline in the total Modified Mayo Score >3 points and >30%.

Induction of remission of ulcerative colitis may be in accordance with the criteria set out in S. P. L. Travis, Aliment Pharmacol Ther 2011; 34: 113-124, the entirety of which is incorporated herein by reference, i.e. complete cessation of rectal bleeding, urgency and increased stool frequency, preferably confirmed by endoscopic mucosal healing.

Alternatively, induction of response or remission may be in accordance with the criteria set out in E. F. Stange, Journal of Crohn's and Colitis (2008) 2, 1-23; S. P. L. Travis, Journal of Crohn's and Colitis (2008) 2, 24-62; K Geboes, Gut 2000; 47: 404-409; the entirety of which are incorporated herein by reference.

The severity of ulcerative colitis and a determination of whether or not a patient is in remission can be measured by reference to one or more standard indices, for instance the Modified Mayo Score discussed above.

The oligonucleotide of the invention is for administration to a patient having inactive ulcerative colitis disease. For instance, the patient may have Modified Mayo sub-scores ≤2, with a rectal bleeding sub-score of 0, a stool frequency sub-score of 0 or 1, and an endoscopy sub-score of 0 or 1. The administration of the oligonucleotide typically extends the period over which the patient has Modified Mayo sub-scores ≤2, with a rectal bleeding sub-score of 0, a stool frequency sub-score of 0 or 1, and an endoscopy sub-score of 0 or 1 for as long as possible, for example for a particular period of time, as defined above.

Prevention of recurrence of or relapse into active ulcerative colitis can be signified by no worsening in any suitable scoring system, for example in the Modified Mayo Score. Typically, the treatment of the present invention results in no worsening in any suitable scoring system, for example in the Modified Mayo Score, for the patient treated for as long as possible, for example for the particular period of time as defined above.

Typically, the treatment of the present invention prevents recurrence of or relapse into active ulcerative colitis as determined by the Modified Mayo Score.

A typical clinical situation presented in the treatment of ulcerative colitis is a patient suffering from an acute/active phase of the disease. The task of the clinician is first to address the acute/active phase, and also to set up a regime to prevent recurrence of or relapse into active ulcerative colitis and/or the symptoms associated therewith for as long as is possible.

Typically, therefore, a patient treated in accordance with the present invention has previously been treated for ulcerative colitis. Preferably, the patient has previously been treated for ulcerative colitis successfully, i.e. the patient treated in accordance with the present invention is typically no longer suffering from the active phase of ulcerative colitis, i.e. the patient is in remission or recovery. Typically, this previous treatment involves administering one or more therapeutic agents which are effective in treating active ulcerative colitis, i.e. inducing remission of active ulcerative colitis. In certain embodiments, the one or more therapeutic agents include the oligonucleotide, particularly cobitolimod. In other embodiments, the one or more therapeutic agents are other than the oligonucleotide and may, for instance, include one or more of the additional therapeutic agents for the treatment of ulcerative colitis defined herein, e.g. may be chosen from immunomodulatory drugs, anti-integrin therapy drugs, TNF-α inhibitors, immunosuppressive drugs, JAK inhibitors or other suitable drugs for treating ulcerative colitis.

Particular examples of suitable drugs include aminosalicylates (such as 5-ASA [also known as mesalamine], sulfasalazine, olsalazine and balsalazide), GCS (such as prednisone, methylprednisolone, hydrocortisone and budesonide), azathioprine (AZA), 6-mercaptopurine (6-MP), tacrolimus, methotrexate, cyclosporine, infliximab (REMICADE®), etanercept (ENBREL ®), adalimumab (HUMIRA®), certolizumab (CIMZIA®), golimumab (SIMPONI®), ustekinumab (STELARA®), vedolizumab (ENTYVIO®), SMAD7 antisense oligonucleotide (Mongersen), natural IFN-β, Decortin, tofacitinib (XELJANZ®), etrolizumab, ozanimod, SHP647, filgotinib, mirikizumab, apremilast (OTEZLA®), estrasimod, AJM300, upadacitinib, risankizumab, BI655130, PF-06700841, PF-06651600 and IMU-83, and equivalents thereof.

The subject treated in accordance with the present invention is typically refractory or responds insufficiently or is intolerant to a therapy. The subject treated in accordance with the present invention is typically refractory or responds insufficiently or is intolerant to an anti-inflammatory therapy. Thus, typically, the subject has previously received or is currently receiving anti-inflammatory therapy, preferably anti-inflammatory therapy for ulcerative colitis. Anti-inflammatory therapies for ulcerative colitis are discussed herein and typically include GCS, TNF-a inhibitors, anti-integrin therapy drugs, aminosalicylates and JAK inhibitors, specific examples of which are listed above.

A refractory disease or disease that responds insufficiently to a therapy is typically a disease where signs and symptoms of active disease persist despite a history of at least one course of the therapy, for example anti-inflammatory therapy.

Intolerance to a therapy, for example anti-inflammatory therapy, means that the therapy has caused side effects in the subject that are not tolerated, e.g. that typically lead to discontinuation of the therapy.

Typically, the subject has previously received or is currently receiving an aminosalicylate, preferably 5-ASA, therapy for ulcerative colitis.

Typically, the subject has previously received or is currently receiving oral, rectal, or parenteral glucocorticosteroids (GCS), preferably oral GCS, therapy for ulcerative colitis.

Typically, the subject has previously received or is currently receiving a TNF-α inhibitor therapy drug for ulcerative colitis.

Typically, the subject has previously received or is currently receiving an anti-integrin therapy, such as vedolizumab, for ulcerative colitis.

Typically, the subject who is refractory or responds insufficiently or is intolerant to an anti-inflammatory therapy shows or has previously shown an inadequate response to, or loss of response to (i.e. is refractory to) or intolerance of oral, rectal, or parenteral GCS treatment (including no GCS treatment due to earlier side effect).

Typically, the subject who is refractory or responds insufficiently or is intolerant to an anti-inflammatory therapy has a history of or current status of an inadequate response (e.g. steroid refractory) to, or steroid dependency, or loss of response to, or intolerance of GCS treatment. The steroids/GCS will typically have been received by the subject in the course of treating ulcerative colitis.

Steroid-refractory typically refers to a subject showing signs and symptoms of persistently active ulcerative colitis despite a history of at least one induction regimen that included a dose equivalent to prednisone 40-60 mg daily orally or prednisolone up to 0.75 mg/Kg/day intravenous (IV) over a period of 4 weeks.

Steroid dependence typically refers to a patient who is either unable to reduce steroids below the equivalent of prednisolone 10 mg/d within 3 months of starting steroids, without recurrent active ulcerative colitis, or who has a relapse within 3 months of stopping steroids.

Intolerance of GCS treatment typically means the subject has experienced Cushing's syndrome, osteopenia/osteoporosis, hyperglycemia, insomnia, infection or other side effects not tolerated by the subject following GCS treatment.

Typically, the patient demonstrates or has previously demonstrated an inadequate response, loss of response, or intolerance to at least one immunomodulator, immunosuppressive therapy, TNF-α inhibitor or anti-integrin. These agents will typically have been administered to the patient to treat ulcerative colitis.

An inadequate response, or loss of response to an immunomodulator typically means signs and symptoms of active ulcerative colitis persist despite previous treatment with an immunomodulator drug such as at least one 8 Week regimen of oral azathioprine (≥1.5 mg/kg) or 6-mercaptopurine (≥0.75 mg/kg).

Intolerance to an immunomodulator typically means the subject has experienced nausea/vomiting, abdominal pain, pancreatitis, liver function test (LFT) abnormalities, lymphopenia, thiopurine methyltransferase (TPMT) genetic mutation, or infection after receiving an immunomodulator.

An inadequate response, or loss of response to a TNF-α inhibitor means signs and symptoms of active ulcerative colitis persist despite previous treatment with a TNF-α inhibitor, such as at least one 4-week induction regimen (or doses as recommended according to the current labels) of infliximab (5 mg/kg (IV), 2 doses at least 2 weeks apart); golimumab (200/100 mg (SC), 2 doses at least 2 weeks apart); or adalimumab (160/80 mg (SC), 2 doses at least 2 weeks apart) or recurrence of symptoms during maintenance dosing following prior clinical benefit.

Intolerance to a TNF-α inhibitor means an infusion-related reaction, demyelination, congestive heart failure, infection, rash or other side effects list above, following receipt of a TNF-α inhibitor.

An inadequate response, or loss of response to an anti-integrin means signs and symptoms of active ulcerative colitis persist despite previous treatment with an anti-integrin, such as at least 10 weeks regimen of vedolizumab 300 mg (IV), or as recommended in the current label, or recurrence of symptoms during maintenance dosing following prior clinical benefit.

In certain embodiments, the patient has previously been treated for active ulcerative colitis using the oligonucleotide, and the oligonucleotide is then also used to prevent recurrence/relapse thereof. The present invention therefore also provides an oligonucleotide as defined herein, in particular cobitolimod, for use in preventing the recurrence of or relapse into active ulcerative colitis in the patient following previous treatment for active ulcerative colitis using the oligonucleotide. In such embodiments, the dosage of the oligonucleotide administered to induce remission and to prevent recurrence of or relapse into active ulcerative colitis may be the same or different.

Typically, the patient treated in accordance with the present invention is susceptible to relapse into or recurrence of the active phase of ulcerative colitis.

The drug is particularly useful in the continuous treatment of patients who are susceptible to recurrence of or relapse into active ulcerative colitis. The oligonucleotide as herein above can be used as maintenance therapy to prevent the recurrence of or relapse into symptoms associated with active ulcerative colitis, and/or to improve the patient's condition.

Typically, the subject has been diagnosed with left-sided ulcerative colitis, i.e. distal colitis, including proctosigmoiditis.

As used herein, the term “oligonucleotide” refers to a polynucleoside formed from a plurality of linked individual nucleoside units. Such oligonucleotides can be obtained from existing nucleic acid sources, including genomic DNA or cDNA, plasmids, vectors, or bacterial DNA, but are preferably produced by synthetic methods. The nucleoside residues can be coupled to each other by any of the numerous known internucleoside linkages. Such internucleoside linkages include, without limitation, the natural internucleoside phosphodiester bond or indeed modified internucleosides such as, but not limited to, phosphorothioate, phosphorodithioate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. The term “oligonucleotide” also encompasses polynucleosides having one or more stereospecific internucleoside linkages (e. g., (Rp)- or (Sp)-phosphorothioate, alkylphosphonate, or phosphotriester linkages). As used herein, the terms “oligonucleotide” and “dinucleotide” are expressly intended to include polynucleosides and dinucleosides having any such internucleoside linkage, whether or not the linkage comprises a phosphate group. In certain preferred embodiments, these internucleoside linkages may be phosphodiester, phosphorothioate, or phosphorodithioate linkages, or combinations thereof.

The term “oligonucleotide” also encompasses polynucleosides having additional substituents including, without limitation, protein groups, lipophilic groups, intercalating agents, diamines, folic acid, cholesterol and adamantane. The term “oligonucleotide” also encompasses any other nucleobase containing polymer, including, without limitation, peptide nucleic acids (PNA), peptide nucleic acids with phosphate groups (PHONA), locked nucleic acids (LNA), morpholino-backbone oligonucleotides, and oligonucleotides having backbone sections with alkyl linkers or amino linkers. The alkyl linker may be branched or unbranched, substituted or unsubstituted, and chirally pure or a racemic mixture.

The oligonucleotides of the invention can include naturally occurring nucleosides, modified nucleosides, or mixtures thereof. As used herein, the term “modified nucleoside” is a nucleoside that includes a modified heterocyclic base, a modified sugar moiety, or a combination thereof. In some embodiments, the modified nucleoside is a non-natural pyrimidine or purine nucleoside, as herein described. In some embodiments, the modified nucleoside is a 2′-substituted ribonucleoside, an arabinonucleoside or a 2′-deoxy-2′-substituted-arabinoside.

As used herein, the term “a hybrid oligonucleotide” is an oligonucleotide having more than one type of nucleoside.

Herein, the term “oligonucleotide” includes hybrid and chimeric oligonucleotides. A “chimeric oligonucleotide” is an oligonucleotide having more than one type of internucleoside linkage within its sequence structure. One preferred example of such a chimeric oligonucleotide is a chimeric oligonucleotide comprising a phosphorothioate, phosphodiester or phosphorodithioate region and non-ionic linkages such as alkylphosphonate or alkylphosphonothioate linkages (U.S. Pat. Nos. 5,635,377 and 5,366,878).

Herein, the term “oligonucleotide” also includes circularized variants and circular oligonucleotides.

Preferably, the oligonucleotide comprises at least one naturally occurring phosphodiester, or one modified phosphorothioate, or phosphorodithioate internucleoside linkage, however preferred linkages or indeed backbone modifications including, without limitation, methylphosphonates, methylphosphonothioates, phosphotriesters, phosphothiotriesters, phosphorothioates, phosphorodithioates, triester prodrugs, sulfones, sulfonamides, sulfamates, formacetal, N-methylhydroxylamine, 2′ OMe (OxyMethyl group at 2′position), carbonate, carbamate, morpholino, boranophosphonate, phosphoramidates, especially primary amino-phosphoramidates, N3 phosphoramidates and N5 phosphoramidates, and stereospecific linkages (e. g., (Rp)-or (Sp)-phosphorothioate, alkylphosphonate, or phosphotriester linkages) are also envisaged.

The sugar moiety of the nucleoside can be a non-naturally occurring sugar moiety. Herein, a “naturally occurring sugar moiety” is a sugar moiety that occurs naturally as part of a nucleic acid, e. g., ribose and 2′-deoxyribose, and a “non-naturally occurring sugar moiety” is any sugar that does not occur naturally as part of a nucleic acid, but which can be used in the backbone for an oligonucleotide, for example but not limited to hexose. Arabinose and arabinose derivatives are examples of preferred sugar moieties.

Modified or substituted oligonucleotides are often preferred over native forms because of desirable properties such as, for example, enhanced cellular uptake, enhanced affinity for nucleic acid target and increased stability in the presence of nucleases. An oligonucleotide is usually comprised of more than ten (10) and up to one hundred (100) or more deoxyribonucleotides or ribonucleotides, although preferably between about eight (8) and about forty (40), most preferably between about eight (8) and about twenty (20). The exact size will depend on many factors, which in turn depends on the ultimate function or use of the oligonucleotide. The oligonucleotide may be generated in any manner, including chemical synthesis, DNA replication, reverse transcription, or a combination thereof.

The oligonucleotide for use in the present invention comprises the sequence 5′-GGAACAGTTCGTCCATGGC-3′ (SEQ ID NO:2). Typically, at least one CG dinucleotide is unmethylated.

Typically, at least one nucleotide in said oligonucleotide has a phosphate backbone modification. The backbone modification is typically a phosphorothioate or a phosphorodithioate modification.

Phosphorothioate linkages can be illustrated with asterisks (*) in a sequence, e.g. in the sequence:
5′-G*G*A*ACAGTTCGTCCAT*G*G*C-3′ (SEQ ID NO:1), wherein the CG dinucleotide is unmethylated.

Preferably, said oligonucleotide has the sequence 5′-G*G*A*ACAGTTCGTCCAT*G*G*C-3′ (SEQ ID NO:1), wherein the CG dinucleotide is unmethylated. Thus, preferably said oligonucleotide is cobitolimod.

The treatments of the present invention typically involve giving multiple doses of said oligonucleotide to a patient to prevent recurrence/relapse for as long as possible, for instance a period of time as defined herein. The multiple doses are typically spaced apart so as to prolong the preventive/protective effect of the oligonucleotide as much as possible, and preferably the individual doses are administered at regular intervals.

Typically, the interval between individual doses is at least 1 week, preferably at least 4 weeks or at least 6 weeks or at least 8 weeks. Typically, the interval between individual doses is up to 12 months, preferably up to 6 months, more preferably up to twelve weeks. For the avoidance of doubt, by “interval” it is meant the period of time between individual doses. Thus, in the case (for example) of a 4 week interval, the duration of time between administration of each individual dose is 4 weeks.

Typically, the interval between individual doses is from 1 week to 12 months, preferably from 4 weeks to 6 months, more preferably from 4 weeks 12 weeks, for example from 4 weeks to 8 weeks or from 8 weeks to 12 weeks.

Typically, the interval between individual doses is 1 week, 2 weeks, 3 weeks, 4 weeks, 6 weeks, 8 weeks, 12 weeks, 6 months or 12 months. Preferably the interval between individual doses is 1 week, 4 weeks, 8 weeks or 12 weeks, more preferably 4 weeks, 8 weeks or 12 weeks, more preferably 4 weeks or 8 weeks. Thus, in a preferred aspect of the invention the interval between individual doses is 4 weeks. In another preferred aspect of the invention the interval between individual doses is 8 weeks.

The above dosage regime, i.e. of administering individual doses of said oligonucleotide to the patient at regular intervals is continued for a time period such that it provides an ongoing maintenance therapy. Given that ulcerative colitis is chronic condition that cannot be cured, this means in practice that patients will generally receive the regimes as described herein for extended periods of time, typically for at least 6 months, preferably for at least 1 year or 2 years, for example for at least 5 year or 10 years. In practice, some patients may need to be administered the regimes as described herein for as long as needed (as determined e.g. by a physician) or indefinitely.

Typically, the individual doses administered in the dosage regimes outlined above are of from 10 mg to 350 mg of said oligonucleotide, preferably from 20 to 300 mg of said oligonucleotide. Typically, the same dosage of oligonucleotide is administered in each individual dose/administration, but different dosages may also be used.

The individual dose of said oligonucleotide, preferably cobitolimod, may be, for instance, from 10 to 50 mg, preferably from 15 to 45 mg, more preferably from 20 to 40 mg, even more preferably from 25 to 35 mg, even more preferably from 26 to 34 mg, even more preferably from 27 to 33 mg, even more preferably from 28 to 32 mg, even more preferably from 29 to 31 mg, especially about 30 mg.

The individual dose of said oligonucleotide, preferably cobitolimod, may be, for instance, from 100 mg to 150 mg, preferably greater than 100 mg up to 150 mg, more preferably from 101 mg to 150 mg, even more preferably from 105mg to 145 mg, even more preferably from 110 to 140, even more preferably from 115 to 135, even more preferably from 120 to 130, even more preferably from 121 to 129, even more preferably from 122 to 128, even more preferably from 123 to 127, even more preferably from 124 to 126 mg, especially about 125 mg

Alternatively, the individual dose of said oligonucleotide, preferably cobitolimod, may be from 150 mg to 350 mg, preferably from 175 mg to 325 mg, more preferably from 200 mg to 300 mg, even more preferably from 210 to 290, even more preferably from 220 to 280, even more preferably from 230 to 270, even more preferably from 240 to 260, even more preferably from 245 to 255, even more preferably from 249 to 251 mg, especially about 250 mg.

In a preferred aspect of the invention, the patient receives multiple doses of the said oligonucleotide at regular intervals, the individual doses of said oligonucleotide are about 30mg, and the interval between the individual doses being administered to the patient is 1 week, 4 weeks, 6 weeks, 8 weeks or 12 weeks, more preferably 4 weeks, 8 weeks or 12 weeks, more preferably 4 weeks or 8 weeks. Thus, in a preferred aspect of the invention, the patient receives multiple doses of the said oligonucleotide at regular intervals, the individual doses of said oligonucleotide are about 30 mg, and interval between those individual doses is 4 weeks. In another preferred aspect of the invention, the patient receives multiple doses of the said oligonucleotide at regular intervals, the individual doses of said oligonucleotide are about 30 mg, and the interval between individual doses is 8 weeks. These regimes are typically continued for at least 6 months, preferably for at least 1 year or 2 years, for example for at least 5 year or 10 years or indefinitely

In a preferred aspect of the invention, the patient receives multiple doses of the said oligonucleotide at regular intervals, the individual doses of said oligonucleotide are about 125 mg, and the interval between the individual doses being administered to the patient is 1 week, 4 weeks, 6 weeks, 8 weeks or 12 weeks, more preferably 4 weeks, 8 weeks or 12 weeks, more preferably 4 weeks or 8 weeks. Thus, in a preferred aspect of the invention, the patient receives multiple doses of the said oligonucleotide at regular intervals, the individual doses of said oligonucleotide are about 125 mg, and interval between those individual doses is 4 weeks. In another preferred aspect of the invention, the patient receives multiple doses of the said oligonucleotide at regular intervals, the individual doses of said oligonucleotide are about 125 mg, and the interval between individual doses is 8 weeks. These regimes are typically continued for at least 6 months, preferably for at least 1 year or 2 years, for example for at least 5 year or 10 years or indefinitely.

In a preferred aspect of the invention, the patient receives multiple doses of the said oligonucleotide at regular intervals, the individual doses of said oligonucleotide are about 250 mg, and the interval between the individual doses being administered to the patient is 1 week, 4 weeks, 6 weeks, 8 weeks or 12 weeks, more preferably 4 weeks, 8 weeks or 12 weeks, more preferably 4 weeks or 8 weeks. Thus, in a preferred aspect of the invention, the patient receives multiple doses of the said oligonucleotide at regular intervals, the individual doses of said oligonucleotide are about 250 mg, and interval between those individual doses is 4 weeks. In another preferred aspect of the invention, the patient receives multiple doses of the said oligonucleotide at regular intervals, the individual doses of said oligonucleotide are about 250mg, and the interval between individual doses is 8 weeks. These regimes are typically continued for at least 6 months, preferably for at least 1 year or 2 years, for example for at least 5 year or 10 years or indefinitely.

In the context of dosage of an active agent, “about” as used herein means +/−10%, typically +/−5%, preferably +/−1%.

In the above dosage regimes individual doses (of an amount as specified herein) of said oligonucleotide are administered to the patient on multiple occasions spaced apart by particular numbers of weeks. This means that the patient does not receive any additional oligonucleotide between the specified doses/administrations divided by the specified time periods.

As used herein, reference to periods of time a number of weeks apart means in certain embodiments administration of the doses exactly that number of weeks apart, e.g. 4 weeks would mean exactly 28 days. However, it will be appreciated that minor variations from this are still within the scope of the present invention. Such minor variations may be unavoidable due to e.g. illness of the patient or unavailability of the drug. Thus, as used herein a period of time a number of weeks apart means that number of weeks +/−3 days, preferably +/−2 days, more preferably +/−1 day. Thus, in this context “4 weeks apart” means administration 25-31 days apart, typically 26-30 days apart, or 27-29 days apart.

The drugs for use in the present invention may be administered as monotherapy treatment for the indication or with other drug(s) as adjunct therapy for the indication, as described in more detail below. In the case of adjunct (or “add-on”) therapy, the drugs for use in the present invention may be administered simultaneously, separately or sequentially with the other drug(s), for example in fixed dose combination or in separate doses.

As used herein, the term “add-on” refers to administering of said oligonucleotide in addition to a current therapy or drug regime, without discontinuing the current therapy or drug regime.

Thus, the oligonucleotide may be administered as a monotherapy, or in combination with one or more additional therapeutic agents for the prevention of recurrence of active ulcerative colitis. Typically, the oligonucleotide may be administered as a monotherapy, or in combination with one or more additional therapeutic agents for the prevention of recurrence of active ulcerative colitis chosen from anti-inflammatory drugs, immunomodulatory drugs, anti-integrin therapy drugs, TNF-α inhibitors, immunosuppressive drugs, JAK inhibitors or other suitable drugs for treating ulcerative colitis or other suitable drugs for treating ulcerative colitis. Examples of such drugs suitable for use in combination with said oligonucleotide include, but are not limited aminosalicylates (such as 5-ASA [also known as mesalamine], sulfasalazine, olsalazine and balsalazide), GCS (such as prednisone, methylprednisolone, hydrocortisone and budesonide), azathioprine (AZA), 6-mercaptopurine (6-MP), tacrolimus, methotrexate, cyclosporine, infliximab (REMICADE®), etanercept (ENBREL®), adalimumab (HUMIRMA®), certolizumab (CIMZIA®), golimumab (SIMPONI®), ustekinumab (STELARA®), vedolizumab (ENTYVIO®), SMAD7 antisense oligonucleotide (Mongersen), natural IFN-β, Decortin, tofacitinib (XELJANZ®), etrolizumab, ozanimod, SHP647, filgotinib, mirikizumab, apremilast (OTEZLA®), estrasimod, AJM300, upadacitinib, risankizumab, BI655130, PF-06700841, PF-06651600 and IMU-83, and equivalents thereof.

For purposes of the invention, the terms “in combination with” and “add-on” mean in the course of treating the same disease in the same patient, and include administering the oligonucleotide and one or more additional therapeutic agents in any order, including simultaneous administration, as well as temporally spaced order of up to several months apart.

Typically, said oligonucleotide is administered topically, such as topically to the mucous membrane.

Typically, said oligonucleotide is administered intracolonically. Intracolonical administration is typically effected rectally. Intracolonical administration is typically effected using an enema or catheter. Intracolonical administration preferably involves administration by an enema (i.e. by rectal enema). Intracolonical administration may be topical, for example performed during colonoscopy with the aid of a spraying catheter, or other suitable medical equipment, inserted though the colonoscopies biopsy channel. The said oligonucleotide may be delivered to the upper portion of the descending colon or to the transverse region of the colon; however other regions are also possible when suited. Topical administration to other parts of the gastrointestinal tract is also possible. Yet in another embodiment of this aspect, the said oligonucleotide can be administered by any appropriate administration route, such as, but not limited to, inhalation, intranasal, parenteral, oral, intradermal, subcutaneous, vaginal and rectal administration. Further, in certain embodiments, systemic administration of said oligonucleotide may be used.

The oligonucleotide may be administered in the form of a pharmaceutical composition comprising the oligonucleotide as defined herein together with one or more pharmaceutically acceptable carriers. As used herein, the term “carrier” encompasses any excipient, diluent, filler, salt, buffer, water, stabilizer, solubilizer, lipid, or other material well known in the art for use in pharmaceutical formulations. It will be understood that the characteristics of the carrier will depend on the route of administration for a particular application.

As used herein, the term “pharmaceutically acceptable” refers to a material that does not interfere with the effectiveness of the immunomodulatory oligonucleotide and is compatible with a biological system such as a cell, cell culture, tissue, or organism. Preferably, the biological system is a living organism, such as a vertebrate.

Typically, the composition is a solution of the oligonucleotide in a liquid carrier, or a foam. Usually, the composition is a solution of the oligonucleotide in a liquid carrier.

Typically, the carrier is water, preferably sterile water. Thus, typically the composition comprises the oligonucleotide as defined herein and water.

Preferably, the carrier is water and the oligonucleotide (in the form of a composition) is administered intracolonically, for instance as a rectal enema.

The oligonucleotide has been found to be advantageously stable in water, and it is therefore possible to administer the oligonucleotide as a composition consisting essentially of the oligonucleotide as defined herein and water. The composition may consist of the oligonucleotide as defined herein and water.

A composition consisting essentially of components refers to a composition comprising the components of which it consists essentially as well as other components, provided that the other components do not materially affect the essential characteristics of the composition.

Typically, a composition consisting essentially of certain components will comprise greater than or equal to 95 wt % (relative to the total weight of the composition) of those components or greater than or equal to 99 wt % (relative to the total weight of the composition) of those components.

Thus, a composition consisting essentially of the oligonucleotide as defined herein and water comprises greater than or equal to 95 wt % of oligonucleotide and water (relative to the total weight of the composition) or greater than or equal to 99 wt % of oligonucleotide and water (relative to the total weight of the composition).

The concentration of an oligonucleotide in a pharmaceutical composition will vary depending on several factors, including the dosage of the oligonucleotide to be administered. Typical concentrations of oligonucleotides in compositions that are solutions are from 0.01 mg/ml to 10 mg/ml, in some instances, 0.1 mg/ml to 6 mg/ml, in some instances from 0.1 mg/ml to 1 mg/ml, in some instances from 2 to 3 mg/ml, in some instances from 4 to 6 mg/ml.

The present invention also provides a pharmaceutical composition comprising an oligonucleotide as defined herein, together with one or more pharmaceutically acceptable carriers, for use in preventing the recurrence of active ulcerative colitis as defined herein in a patient as defined herein.

Preferred features of the oligonucleotide for use as defined above are also preferred features of the composition for use.

The present invention also provides use of an oligonucleotide as defined herein, or a pharmaceutical composition as defined herein, in the manufacture of a medicament for use in preventing recurrence of active ulcerative colitis as defined herein, in a subject as defined herein.

Preferred features of the oligonucleotide for use as defined above are also preferred features of the use of the oligonucleotide or composition.

The present invention also provides a method of preventing recurrence of active ulcerative colitis as defined herein, in a subject as defined herein, comprising administering to said subject an oligonucleotide as defined herein or a composition as defined herein.

The present invention also provides the use of an oligonucleotide as defined herein, for the manufacture of a medicament for preventing the recurrence of active ulcerative colitis in a patient as defined herein.

Preferred features of the oligonucleotide for use as defined above are also preferred features of the use of the oligonucleotide.

The following non-limiting Examples illustrate the invention.

### Example 1—Clinical Trial Study

In this randomized, double-blind, placebo-controlled trial, 131 patients with moderate-to-severe active UC were randomized to receive two single doses of the oligonucleotide cobitolimod [30 mg] or placebo administered topically during lower GI endoscopy at baseline and Week 4. The primary endpoint was clinical remission, defined as Clinical Activity (Rachmilewitz) Index [CAI]≤4, at Week 12. Secondary endpoints included mucosal healing and symptomatic remission of key patient-reported outcomes [absence of blood in stool and weekly stool frequency <35].

Eligible patients were adults with moderate to severely active ulcerative colitis with a Clinical Activity Index [CAI] of ≥9 and an endoscopic Mayo score of ≥2, despite treatment with glucocorticosteroids [GCS] [prednisolone] equivalent ≥10 mg/day] for ≥2 weeks prior to inclusion, and previously having failed or been intolerant to treatment with mesalazine ≥2.4 g/day for ≥4 weeks, GCS with at least 0.75 mg/kg as a starting dose, azathioprine or mercaptopurine for ≥3 months, and/or one adequate treatment course of a TNF-α inhibitor. Patients may have tried treatment with cyclosporine or tacrolimus before the trial. Four patients in the placebo group [9.3%] and 5 patients in the cobitolimod group [6.2%] had used cyclosporine prior to the study start, but not in the study.

During the trial, patients could be taking sulfasalazine, aminosalicylates, or thiopurines, and should be on oral GCS at stable doses. Concurrent therapies with cyclosporine, tacrolimus or TNF-α inhibitors during the trial or in the 4 weeks before enrolment, or antibiotics or non-steroidal anti-inflammatory drugs [NSAIDs] in the 2 weeks before enrolment were not permitted. Patients were excluded if they had a current diagnosis of fulminant colitis, indication for immediate surgery, signs of active infection [temperature ≥38° C.], or haemoglobin <100g/L. Additional exclusion criteria were current parenteral nutrition, blood transfusion, C. difficile infection, current or past colonic malignancy and/or dysplasia, clinically significant compromise of major organ function, and concurrent or previous use of investigational therapy up to 30 days before enrolment.

Women who were pregnant or breast-feeding were excluded.

Eligible patients were randomized in a 2:1 ratio to receive administration of cobitolimod via endoscopy [30 mg] at Week 0 and 4, or matching placebo diluted in 50 mL of sterile water after adequate bowel cleaning for stool content. The application was done proximally to the site of mucosal inflammation, or in the transverse section of the colon in the event of extensive colitis, using a spray catheter during endoscopy. Patients were asked to remain recumbent for 2 h after administration. Patients were followed up with visits after 1, 4, 8, 12, 22, and 52 weeks. At Weeks 0, 4, and 12, patients underwent endoscopic evaluation and biopsies were taken from the most inflamed mucosal area. ICON [Texas, USA] produced the randomization code by a computer-generated procedure, which used the method of randomly permuted blocks. Oral GCS treatment was mandatory at a stable daily dose of >10 mg initiated at least 2 weeks prior to inclusion. Steroid tapering was done to a standard tapering schedule according to European Crohn's and Colitis Organisation [ECCO] guidelines when the patient had reached clinical remission, the earliest at Week 12.

### Endpoints

The primary endpoint was induction of clinical remission at Week 12, defined as a CAI score of ≤4. This endpoint was chosen since clinical benefit using this measure was observed in previous compassionate use. Secondary endpoints included mucosal healing [endoscopic Mayo sub-score ≤1, endoscopies not centrally read]; clinical remission at Weeks 1, 4, 8, 22 and 52; clinical remission and mucosal healing; symptomatic remission [defined as absence of blood in stool and weekly stool frequency of <35]; histological Geboes score at Week 4 and 12 as assessed by a single trial histopathologist; time to colectomy; and quality of life based on the inflammatory bowel disease questionnaire [IBDQ] and the 36-item short-form survey [SF36] score. Post hoc, the combined score for subjects who achieved both symptomatic remission and mucosal healing was defined.

### Safety Evaluations

Safety and tolerability were evaluated throughout the study. Safety was assessed by vital signs, ECG results, laboratory variables, and adverse event [AE] reports. Patients were free to discontinue their participation in the study at any time or could be withdrawn from study treatment at the discretion of the investigator.

### Results and Conclusions

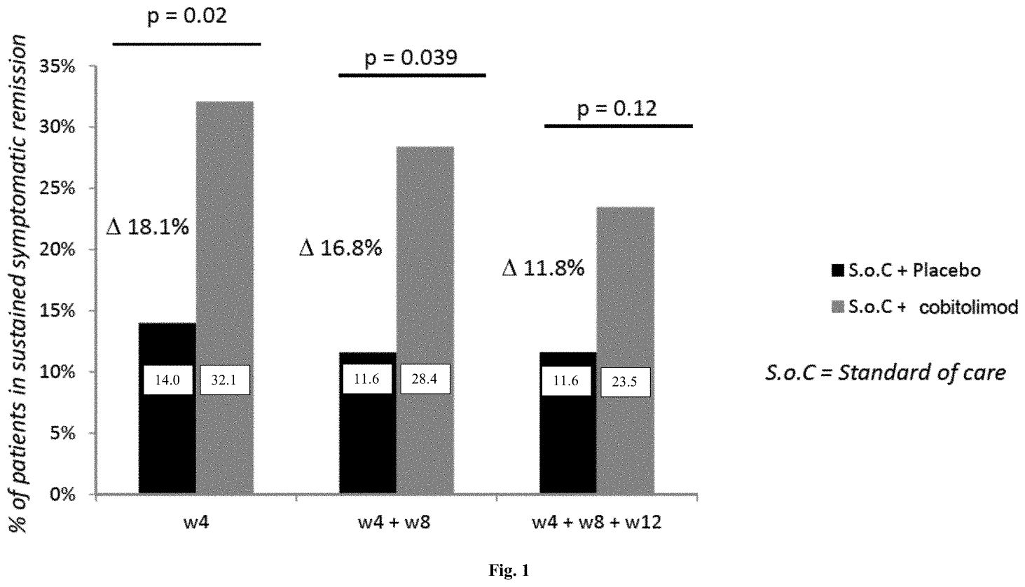

FIG. 1 shows the percentage of patients in symptomatic remission (as evidenced by blood in stool and stool frequency) at week 4, with information on how that percentage changes for that same group of patients at weeks 8 and 12.

The 32.1% of patients administered 30 mg cobitolimod at week 0 were in symptomatic remission at week 4 (delta of 18.1% between active drug and placebo). As discussed above, these patients were administered another 30 mg dose of cobitolimod at week 4. By week 8, 28.4% of the patients were in remissions (a decrease of 3.7% as compared to the percentage age week 4) (delta of 16.8% between active drug and placebo). The patients were not administered a further dose of cobitolimod at week 8. By week 12, 23.5% of patients were in remission (a decrease of 4.9% as compared to the percentage at week 8) (delta of 11.8% between active drug and placebo).

These results show that following induction of remission by the dose of cobitolimod at week 0, there is a tendency for the patients to relapse into the active phase of ulcerative colitis. However, the tendency of the patient to relapse into the active phase of ulcerative colitis is reduced by administration of a further dose of cobitolimod which was not the case for placebo treated patients. That is evident from the results at week 8 as compared to week 12. The patients at week 8 have benefited from the further dose of cobitolimod at week 4 (i.e. 4 weeks earlier), and so fewer patients relapse into the active phase of ulcerative colitis. In contrast, at week 12 the patients have not received a further dose of cobitolimod 4 weeks earlier (i.e at week 8), and so more patients relapse into the active phase of ulcerative colitis

It can be concluded from these results that patients in remission from active ulcerative colitis (for example following induction of remission by administration of cobitolimod) tend to relapse into the active phase of ulcerative colitis, unless provided with further doses of cobitolimod. These further doses of cobitolimod serve to keep patients in remission and prevent relapse into the active phase of ulcerative colitis. Accordingly, repeated dosing with cobitolimod once remission has been induced maintains patients in remission and prevent relapse into the active phase of ulcerative colitis.

### Example 2—Clinical Data from Compassionate Use Program

This example relates to unpublished experimental data that was collected as part of a compassionate use program. A diagnosis of ulcerative colitis was established based on clinical, endoscopic and histological features. Written consent was received for the patient and the treatment approved by the relevant local ethics committee.

The patient was a 49 year old male who was first diagnosed with ulcerative colitis 5 years previously and who had become refractory to steroids and immunosuppressive therapies (GCS, infliximab and natural IFN-β). In the form of an experimental add-on therapy, relapses were treated for 4 years with natural Interferon-beta (Fiblaferon). This treatment had to be stopped because the insurance company did not renew the payment agreement. A severe relapse in January 2010 was treated with high doses of steroids in combination with azathioprine and 5-ASA without success. The patient went on to receive one dose of Infliximab at the end of January but this lead to a severe salmonellosis with septicemia in February 2010. The treatment with a high dose of steroid in combination with Infliximab had some effect in reducing the CAI score from 14 to 9 in March 2010. However, the subject was still in a very active state of the disease.

Therefore, a multi-dose treatment regime with cobitolimod was considered and the first dose given on the 26 of Mar. 2010, with 2 additional doses administered 4 weeks apart.

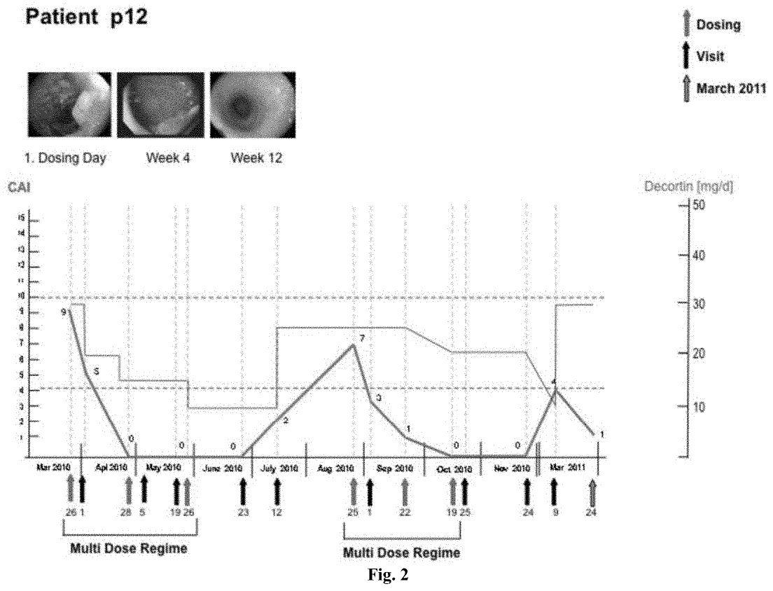

The patient received 3 doses of 30 mg cobitolimod diluted in 50 mL of sterile water at 4 week intervals as an add on to current therapies. Application of drug was performed during colonoscopy with the aid of a spraying catheter inserted through the colonoscopies biopsy channel and delivered approximately to the upper portion of the descending colon or to the transverse region. After one week, the patient had a drop in the CAI score of 4 points and nearly reached clinical remission (see FIG. 2).

The physician decided to start a very slow steroid tapering regime due to the fact that cobitolimod was to be given 2 more times. At week 4, the time point of the second cobitolimod instillation, the patient had reached complete remission with continued steroid tapering. Complete clinical remission was maintained for a further two months while receiving the last dose of cobitolimod and further steroid tapering.

Upon signs of deterioration (increasing CAI score) it was decided to treat this relapse with another multi-dosing regime of cobitolimod that was started on the 25 of Aug. 2010.

This time clinical remission was reached within 4 weeks and by week 8, the patient was in complete clinical remission again that was maintained for 5 months until March 2011 (see FIG. 2).

FIG. 2 shows the treatment course of patient p12. Depicted is the colitis activity index [CAI] against the time in months. A CAI score with a value above the upper dashed line shows that the patient has a severe flare in the disease and below the lower dashed line that the patient is in clinical remission. The solid line without numbers shows the course of the steroid treatment with the amount of Decortin [mg] was given per day. A grey arrow indicates a dosing visit (26 Mar. 2010, 28 Apr. 2010, 26 May 2010, 25 Aug. 2010, 22 Sep. 2010, 19 Oct. 2010), a black arrow a visit where a CAI score could be established (1 Apr. 2010, 5 May 2010, 19 May 2010, 23 Jun. 2010, 12 Jul. 2010, 1 Sep. 2010, 25 Oct. 2010, 24 Nov. 2010, 9 Mar. 2011) and the grey arrow on the right hand side shows the status on 24 Mar. 2011 when the treating physician last contacted all the patients to follow-up on the treatments.

The CAI scores in FIG. 2 show that once the patient is in remission (CAI<4) following the first dose of cobitolimod, further repeat dosing with cobitolimod maintains the patient in remission and prevents relapse of the disease. In the first multi-dose regime for patient p12, the repeat dosing with cobitolimod kept the patient in remission for 3 months (May, June and July). Similarly for the second multi-dose regime for patient p12, the repeat dosing kept the patient in remission for over five months (until March 2011). From this data, it is expected that if further doses of cobitolimod were administered at regular intervals, remission could be maintained and relapse into the active phase of ulcerative colitis prevented. It is noteworthy that once cobitolimod dosing stopped, after a few months the patient relapsed into an active disease state. The data in FIG. 2 show that if cobitolimod dosing were maintained, the CAI score could be suppressed and the patient kept in a state of remission. Accordingly, repeated dosing with cobitolimod once remission has been induced maintains patients in remission and prevents relapse into the active phase of ulcerative colitis.

### Example 3—Mouse Studies in DSS Model of Chronic UC

The objective of this study was to test the efficacy of cobitolimod as a maintenance therapy in the chronic DSS-induced mouse model of colitis.

### Animals

37 male C57BL/6 mice were enrolled in the chronic DSS-induced colitis study and housed for a stabilization period of 5 days prior to the study. All animals were housed in HEPA-ventilated cages with sani-chips bedding. Domes and sterile cotton scratch pads were provided for enrichment. Fluorescent lighting was provided on a 12-hour cycle. Temperature and humidity were monitored and recorded daily and maintained to 20-23° C. temperature, and 30-70% relative humidity. 2018S 18% protein rodent maintenance diet was provided ad libitum.

### Drug Formulation

Cobitolimod was diluted in saline either to 125 μg/100μ1 (1.25 μg/μl, dose 1) or 500 μg/100 μl (5 μg/μl, dose 2) in the beginning of the experiment. The solution was aliquoted and kept at 4° C. for the duration of the experiment. At each administration day 100 μl was administered to the mice rectally. FTY-720 (S1P1 receptor inhibitor) was dissolved in dH2O and served as positive control. For a dose of 1 mg/kg, using an average body weight of 30 grams, the compound was prepared and 100 μL orally dosed daily to each animal.

A dose of 500 μg in mice is approximately equivalent to a 125 mg dose in a human (see “Guidance for Industry—Estimating the Maximum Safe Starting Dose in Initial Clinical Trials for Therapeutics in Adult Health Volunteers”, US Department of Health and Human Services Food and Drug Administration Center for Drug Evaluation and Research, July 2005). The dose of 125 μg in mice is broadly equivalent to a dose of 30 mg in humans. The results observed in this mouse model could be considered predictive of the effects observed in humans administered with individual doses of 125 mg or 30 mg of cobitolimod.

### Experimental Design for DSS Induced Chronic Colitis

9 week old mice received 2% DSS dissolved into autoclaved water ad libitum (Group 2-5, n=8 each per group) for 5 days, and were switched to autoclaved drinking water for the next five days. This cycle was repeated, alternating between DSS treatment and autoclaved water, every five days for a duration of 30 days in total. During each 5-Day DSS treatment phase, fresh 2% DSS was provided on the third day of the five-day cycle. Group 1 (n=5) served as a control (naïve mice) that received only autoclaved water ad libitum throughout the study. On Day 4, mice in Group 3 (n=8) were orally dosed with FTY-720 as positive control (Group 3, n=8) and continued daily dose until end of the study. Also starting on Study Day 4, animals were dosed with Vehicle (Group 2, n=6), or with two different doses of cobitolimod (Group 4 and Group 5, n=8 per group) intrarectally. For group 2, n =6 because two mice in group 2 failed to respond in the expected way to the DSS treatment, i.e. were not compatible with the UC model used and were omitted from the analysis. Groups 2, 4, and 5 were dosed again on Days 8, 12, 16, 20, 24, and 28.

TABLE 4
treatment arms in mouse study
Study group

Group 01: Naïve Mice
Group 02: 2% DSS + Vehicle (Saline)
Group 03: 2% DSS + FTY720
Group 04: 2% DSS + Cobitolimod 125 ug
Group 05: 2% DSS + Cobitolimod 500 ug

Clinical and Histological Observations

Body weight, stool consistency, stool blood, body condition score, and physical health were recorded on dosing days, and two days after each dosing day from at Day 0 through termination on Study Day 30. Animals were observed for changes in physical health on scoring/dosing days, and daily if body weight loss of >20% was observed. Animals that lost greater than 20% of their pre-treatment body weight were monitored and fed diet/hydro gel for 72 hrs. No animals were terminated due to prolonged body weight loss, or signs of pain and distress due to disease.

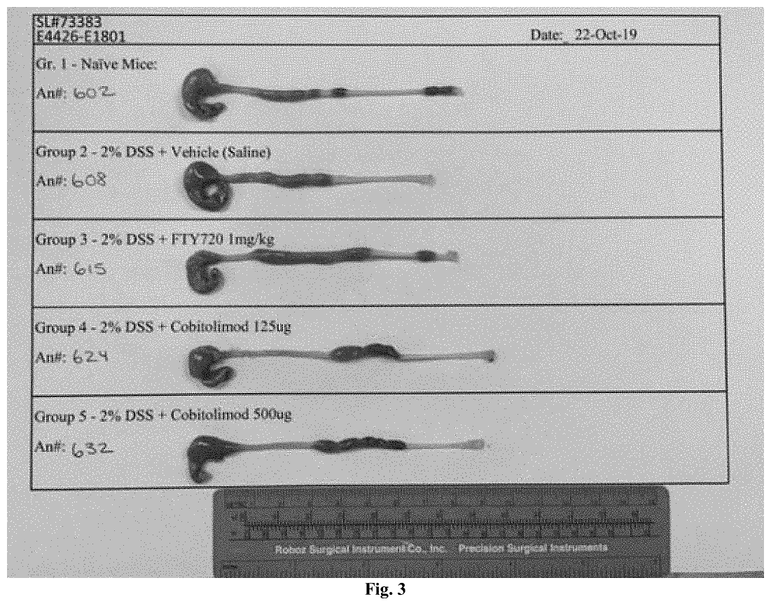

The study was terminated on Day 30, and necropsy was performed on all animals. Spleens were weighed and then discarded. Colon length and weight was recorded, and the weight/length ratio calculated for each animal. A photograph was taken of one representative colon from each group of animals (both control and treatment groups—see FIG. 3). Statistical analysis was performed using GraphPad Prism software.

### Results

The chronic forms of colitis, which more accurately reflect the long-lasting and relapsing nature of IBDs in humans, are more suitable to evaluate the drug efficacy in maintenance therapy. In the chronic experimental mouse model, the animals receive DSS treatment for a long period. Orally received DSS causes death of epithelial cells, compromises barrier function and causes subsequent inflammation. Therefore a lower percentage of DSS in the drinking water is usually used in the chronic model.

In the present chronic experimental mouse model only 2% DSS was used and the DSS treatment and autoclaved water were alternated every 5 days. As a consequence, the clinical symptoms observed were very mild. The mice lost at most 5% of their body weight throughout the study, therefore it is difficult to distinguish the drug effect in the clinical endpoints. However, the most obvious difference between the active and placebo treatments became evident when the colon lengths were measured after the animals were euthanised. Reduction in the length of the colon is a cardinal sign of IBD. It has been reported by Cooper et al. that the length of the colon is inversely linked to the severity of DSS-induced colitis (H S Cooper, S N Murthy, R S Shah, D J Sedergran, Clinicopathologic study of dextran sulfate sodium experimental murine colitis. Laboratory investigation. 1993, Vol. 69(2), p. 238-249). In the absence of effective treatment, the colon length is reduced and significant increase in the relative colonic weight/length ratio is observed in DSS treated animals compared to control animals.

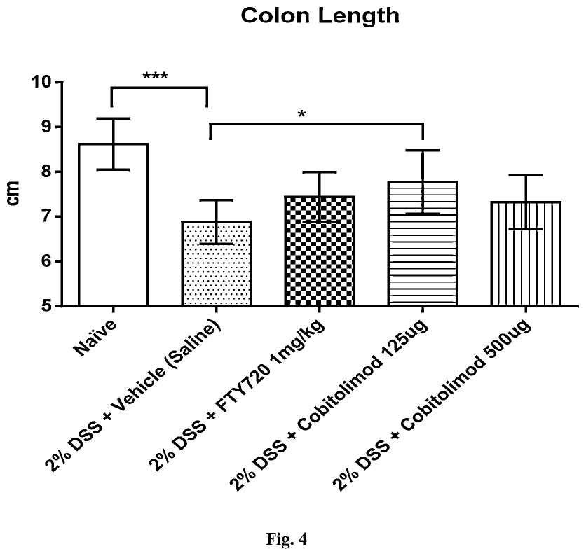

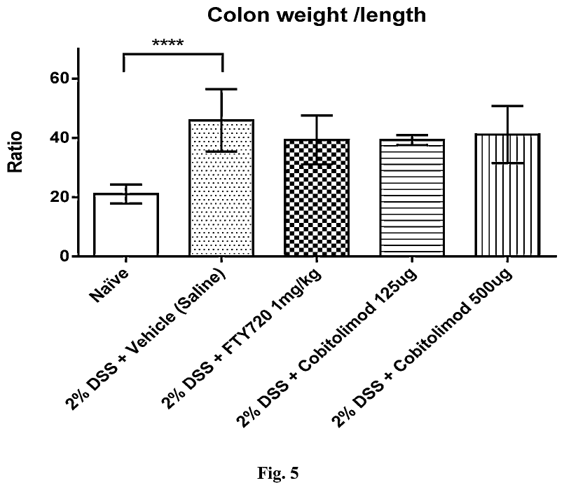

In the present study, treatment with the 125 μg dose of cobitolimod significantly reduced the colon-shortening effects of DSS (see FIGS. 3, 4 and 5). Treatment with the 500 μg dose of cobitolimod also reduced the colon-shortening effects of DSS. This indicates that cobitolimod is able to mitigate the inflammatory effects of the DSS in the chronic UC model. In particular, repeated dosing with cobitolimod was able to reduce colon shortening (i.e. maintain a healthier colon length) following extended exposure to DSS. The colonic weight/length ratio increased significantly in the DSS treated animals compared to naive animals (FIG. 5). However, treatment with both doses of cobitolimod decreased the weight/length ratio (FIG. 5), also indicating that cobitolimod is efficacious in preventing inflammation.

These results show that cobitolimod is not only able to provide initial treatment for the symptoms caused by DSS, but is also able to act as a longer-term therapy to continually suppress inflammation, as shown by the physical differences in the colon between mice receiving cobitolimod and those that did not. Thus, cobitolimod can be used to keep patients in remission and prevent relapse into the active phase of ulcerative colitis. Accordingly, repeated dosing with cobitolimod once remission has been induced maintains patients in remission and prevents relapse into the active phase of ulcerative colitis.

1. A method for preventing the recurrence of active ulcerative colitis in a patient, comprising administering to said patient an oligonucleotide, wherein the oligonucleotide comprises the sequence
(SEQ ID NO: 2)
5′-GGAACAGTTCGTCCATGGC-3′.

2. The method according to claim 1, wherein at least one CG dinucleotide in said oligonucleotide is unmethylated.

3. The method according to claim 1, wherein at least one nucleotide in said oligonucleotide has a backbone modification, and preferably wherein said backbone modification is a phosphate backbone modification represented by a phosphorothioate or a phosphorodithioate modification.

4. The method according to claim 3, wherein said backbone modification is located in the 5′- and/or the 3′-ends of said oligonucleotide.

5. The method according to claim 1, wherein said oligonucleotide has the sequence 5′-G*G*A*ACAGTTCGTCCAT*G*G*C-3′ (SEQ ID NO:1), wherein the CG dinucleotide is unmethylated.

6. The method according to claim 1, wherein said oligonucleotide is cobitolimod.

7. The method according to claim 1, wherein the patient has previously been treated for active ulcerative colitis.

8. The method according to claim 1, wherein the patient has previously received one or more therapeutic agents for the treatment of active ulcerative colitis.

9. The method according to claim 8, wherein the one or more therapeutic agents includes an oligonucleotide comprising the sequence 5′-GGAACAGTTCGTCCATGGC-3′ (SEQ ID NO:2).

10. The method according to claim 1, which oligonucleotide is for use as a maintenance therapy.

11. The method according to claim 1, wherein the patient is refractory or responds insufficiently or is intolerant to an anti-inflammatory therapy.

12. The method according to claim 1, wherein individual doses of said oligonucleotide are administered to the patient with regular intervals between those individual doses.

13. The method according to claim 12, wherein the individual doses are doses of from 10 mg to 350 mg of said oligonucleotide or wherein the individual doses are doses of about 30 mg, or of about 125 mg or of about 250 mg of said oligonucleotide.

14. (canceled)

15. The method according to claim 13, wherein the interval between individual doses is from 1 week to 12 months.

16. The method according to claim 15, wherein the interval between individual doses is 1 week, 4 weeks, 6 weeks, 8 weeks, 12 weeks, 6 months or 12 months.

17. (canceled)

18. The method according to claim 12, wherein the patient is administered the individual doses at regular intervals for at least 6 months, preferably for at least 1 year or 2 years, more preferably for at least 5 years or 10 years or indefinitely.

19. The method according to claim 1, wherein the oligonucleotide is administered as a monotherapy.

20. The method according claim 1, wherein the patient receives one or more additional therapeutic agents for preventing the recurrence of active ulcerative colitis.

21. The method according to claim 1, wherein said oligonucleotide is administered topically to mucosal membranes, preferably wherein said oligonucleotide is administered rectally.

22. A method for preventing the recurrence of active ulcerative colitis in a patient, comprising administering a pharmaceutical composition comprising an oligonucleotide, wherein the oligonucleotide comprises the sequence 5′-GGAACAGTTCGTCCATGGC-3′ (SEQ ID NO:2), together with one or more pharmaceutically acceptable carriers.

23-25. (canceled)
